(19) United States
(12) Patent Application Publication
(10) Pub. No.: US 2007/0223800 A1
(43) Pub. Date: Sep. 27, 2007

(54) METHOD AND SYSTEM FOR VIRTUAL SLICE POSITIONING IN A 3D VOLUME DATA SET

(76) Inventor: Jens Guehring, Monmouth Junction, NJ (US)

Correspondence Address: SCHIFF HARDIN, LLP;PATENT DEPARTMENT, 6600 SEARS TOWER, CHICAGO, IL, 60606-6473, US

(21) Appl. No.: 11/688,993

(22) Filed: Mar. 21, 2007

(30) Foreign Application Priority Data
Mar. 21, 2006 (DE) 10 2006 012 945.8

(51) Int. Cl. H05G 1/60; G06K 9/00; A61B 6/00; A61B 5/05; G01N 23/00; G21K 1/12

(52) U.S. Cl. 382/131; 378/4; 600/410

(57) ABSTRACT

In a method and system for virtual slice positioning in a 3D volume data set in which the image of a subject is represented, first features are extracted from the 3D volume data set that are associated with the subject. An interdependency is determined between the 3D volume data set of the subject and a reference system that corresponds to the 3D volume data set, by setting extracted first features in relation to corresponding second features in the reference system. A first slice positioning that is predefined at the reference system is transferred to a second slice positioning in the 3D volume data set using the determined interdependency. Image data are generated from the 3D volume data set along the second slice positioning (43).

### BACKGROUND OF THE INVENTION

1. Field of the Invention

The present invention concerns a method for virtual slice positioning in a 3D volume data set with the aid of a reference data set and a medical imaging system.

2. Description of the Prior Art

In medical imaging there are various modalities with which a 3D volume data set of a subject can be acquired, for example computed tomography (CT) and magnetic resonance tomography (MRT).

Such methods typically require both the acquisition of data and (in the case of evaluation of the data) interaction with a user who, using methods running semi-automatically, influences the further method progression by his or her interaction. A user is often occupied for a long time with the implementation of the method due to the interaction, and the result of the method is dependent on the type and manner of the interaction, which can vary depending on the user.

It is therefore generally sought to largely automate existing methods. One possibility for automation in the framework of the acquisition of data is disclosed in U.S. Pat. No. 6,195,409. This method serves for automatic slice positioning given the acquisition of the 3D volume data set. After an overview image (which is quickly generated) has been scanned, the image information so acquired is automatically correlated with a reference image. A slice position (previously established at the reference image) that is adapted to the medical question can thus be adapted to the subject to be examined with the aid of the determined correlation. The image data are thereupon acquired along the transferred slice positions. A standardized slice positioning is thus obtained in an automated manner although the subjects to be examined exhibit differences from individual to individual. In addition to other methods, an analogous method is disclosed in US 2003/139659 A1 in which subsequently acquisitions can likewise be controlled based on data of an atlas of the subject to be examined.

DE 199 43 404 A1 also concerns the automation of method steps in the acquisition of data. Here a diagnostic question is selected by a user after a rough positioning of a patient. Specific anatomical landmarks are subsequently determined automatically dependent on the selection and measurement parameters based thereupon are established for subsequent MR measurements.

Common to these methods is the possibility of an automatic slice selection in the data acquisition. Among other things, this is advantageous when successive measurements are implemented, for example in order to track the course of an illness. A largely constant spatial orientation of the slices is provided by the automated slice positioning, such that images acquired at different points in time can be compared without reformatting.

It is not always possible, however, to implement the automatic slice positioning before a measurement. In practice it normally occurs that not all acquisition systems have been fitted with this feature, so patient data sets are acquired whose slice positioning does not correspond. Even if the acquisition system embodies automatic slice positioning, in specific situations it can occur that the automatic slice positioning is not used—for example given an incorrect control instruction, or in an emergency situation in which the automatic slice positioning is foregone in favor of a faster image acquisition. In the event that the medical question changes during the course of the illness of the patient, it can occur that a different automatic slice positioning is selected dependent on this change.

In each of these cases data sets are generated with which a comparison with data sets acquired at different points in time is problematic.

This problem has conventionally been addressed by the user attending to whether the representation of two data sets acquired at different times is comparable at all, and furthermore (given only slight deviations of the representations) takes this deviation into account in the evaluation. The interpretation of the results is thereby made more difficult, and represents a high demand on the attention of the user.

### SUMMARY OF THE INVENTION

An object of the present invention is to provide a method with which a virtual slice positioning can be implemented retroactively in an acquired 3D volume data set in order to compensate for deviating representations of two data sets. A further object of the invention is to provide a medical imaging system with which an image can be automatically evaluated.

The first of the above object is achieved in accordance with the present invention’ by a method for virtual slice positioning in a 3D volume data set in which the image of a subject is stored, including extraction from the 3D volume data set of first features that are associated with the subject, determination of an interdependency or correlation or interrelation between the 3D volume data set of the subject and a reference system that corresponds to the volume data set, by setting the extracted first features are set in relation to corresponding second features in the reference system, transfer of a first slice positioning that is predefined at the reference system to a second slice positioning in the 3D volume data set using the determined interdependency, and generation of image data from the 3D volume data set along the second slice positioning.

The reference system is thereby adapted to the subject stored in the 3D volume data set. Since the reference system can be a generalized (and thus also idealized) stored representation of the subject, the first slice positioning can predefined particularly precisely, robustly and simply at the reference system. This first slice positioning is then transferred to the 3D volume data set using the determined interdependency. The first slice positioning is thus adapted to the individual particulars of the 3D volume data set and of the subject represented therein.

The interdependency is determined by features of the subject and corresponding features of the reference system being set in relation to one another. Which specific features these are depends on the subject to be imaged, the reference system and the type of the 3D volume data set. They are typically prominent features that can be particularly easily located in the image data set or in the reference system and extracted from this. The features should likewise not exhibit excessively large differences between various objects of the same type. When the features satisfy these conditions, the algorithms that are used for location and extraction of the features can be fashioned relatively simply.

The features that originate from the reference system are typically not newly extracted with each implementation of the inventive method. For example, it can be sufficient to identify the prominent features in the reference system once, and to locate the corresponding features in the image data set upon implementation of the method.

Using the method it is now possible to adapt the first slice positioning (which has been precisely defined once at the reference system) to a 3D volume data set and to the subject represented therein without the user having to manually and/or semi-automatically adapt the slice positioning to the individual particulars of the subject.

The subject is preferably a human or animal body or a portion thereof. Particularly in medical imaging, a plurality of 3D volume data sets are often produced at different points in time, for example in order to monitor the progression of an illness. If the correct slice orientation is not specifically adhered to in the data acquisition, the generated 3D volume data sets cannot be directly compared with one another. When a reformatting of the 3D volume data sets is undertaken after the event in order to obtain comparable slice images, conventionally this had to be implemented manually. This is now possible in an automatic manner with the inventive method that is applied in an advantageous embodiment to medical 3D volume data sets.

Various systems that can image the subject in a generalized (and thereby idealized) form are suitable as a reference system. For example, a coordinate system with anatomical features of an organ to be imaged can serve as a reference system. Such a coordinate system is, for example, used in a Talairach system that describes the human brain. In addition to a coordinate system, in the Talairach system a number of planes are described that also can be located relatively simply in an image of the brain. This enables the setting of an image of a real brain and the standard brain described in the Talairach system relative to one another in a simple manner.

It is also possible to use an atlas of the body part to be imaged as a reference system. Such an atlas can be generated, for example, from the imaging of one or more healthy control persons as is, for example, described in United States Patent Application Publication No. 2003/0139659 A1.

In a reference system that is particularly simple to generate, only one 3D volume data set of a control person serves as a reference system. This control person preferably exhibits no anatomical peculiarities.

The reference system thus does not have to exhibit all features that are also to be found in a 3D volume data set of the subject. In general it is sufficient for the reference system to exhibit all features that are necessary for identification of the interdependency and is detailed such that the first slice position can be defined with sufficient precision. For example, for a simple organ to be imaged it can be sufficient for the reference system merely to exhibit the contour of the organ.

The interdependency is preferably described by a rigid, affine or non-linear transformation. The selected type of transformation is thereby adapted to the medical question and the organ system to be imaged and represents a compromise between precision of the relation and calculation time for determination of the relation.

In an embodiment the interdependency is determined by a comparison of characteristic landmarks in the 3D volume data set and in the reference system. Such anatomical landmarks typically represent prominent characteristics in the 3D volume data set, which can therefore be located relatively easily, The transformations and interdependencies between the 3D volume data set and the reference system can be derived simply by a comparison of anatomical landmarks, in particular their size and spatial position.

In another preferred embodiment the interdependency is determined by a comparison of intensity distributions in the 3D volume data set and in the reference system.

The generation of image data from the 3D volume data set preferably ensues along the second slice positioning by multiplanar reformatting.

The predefined slice positioning in the reference system is advantageously established dependent on a medical question, For this purpose, the predefined slice positioning is selected from a pool of multiple different predefined slice positionings. In this manner a user can start the method (for example via input of the symptoms, for example hemiparesis of the left side) by the predefined slice positioning that matches the symptoms (in this case a slice positioning that particularly advantageously covers the motor cortex) being established. For medical imaging systems in which an automatic slice positioning can be implemented before an acquisition to be executed, the predefined slice positionings stored for this purpose can also be used in order to implement a retroactive slice positioning in a 3D volume data set.

In a preferred embodiment the predefined slice positioning is modified using an input of parameters. This is in fact not necessary since the inventive method is designed for an automatic execution, but the method thereby gains additional flexibility.

In a preferred embodiment the 3D volume data set is a 3D volume data set acquired with a computed tomography apparatus or with a magnetic resonance tomography apparatus.

The inventive medical imaging system is equipped with a computer that is fashioned for implementation of the method as described above.

### DESCRIPTION OF THE DRAWINGS

FIG. 1 illustrates a reference model with a first slice positioning adapted to the medical question.

FIG. 2 illustrates an acquired 3D volume data set in which the image of an organ is stored.

FIG. 3 illustrates corresponding features between the reference model and the image of the organ, from which the transformation is determined that sets the reference model in relation to the image of the patient and vice versa, in accordance with the invention.

FIG. 4 illustrates the adaptation of the first slice positioning to the image stored in the 3D volume data set using the determined transformation.

FIG. 5 is a flowchart of an embodiment of the inventive method.

### DESCRIPTION OF THE PREFERRED EMBODIMENTS

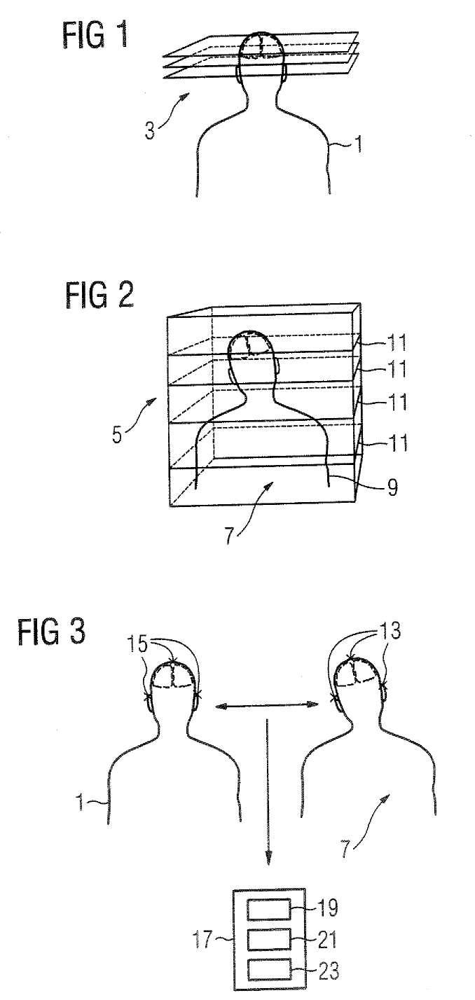

A reference body 1 is shown in FIG. 1. A first slice positioning 3 can be defined particularly precisely and simply at such a reference body 1, which is free of individual peculiarities. The slice positioning 3 is thereby typically adapted to a specific medical question.

The first slice positioning 3 drawn in FIG. 1 is transversally oriented in order to image the brain at a specific level that is particularly advantageous for a specific medical question (for example stroke diagnosis).

As illustrated above, such predefined slice positionings are used in the planning of the measurement parameters given an MRT or CT examination, as described in U.S. Pat. No. 6,196,409 and DE 199 43 404 A1.

By contrast, FIG. 2 shows a 3D volume data set 5 in which the image 7 of a patient 9 is not stored in an ideal position. Such deviations from an ideal position are the rule in practice and can be ascribed to various causes, for example to an incorrect positioning of the patient 9 or to an imprecise positioning of the patient 9 in an image data acquisition system.

The slice orientation in the 3D volume data set 5, indicated by a few horizontal slices 11, is such that the slice images (which should be actual transversal slice images of the patient 9) intersect the head at an angle. The evaluation of these images represents a significant challenge for the user since he or she must take the angled slice direction (which, in terms of its magnitude, cannot be determined without further measures using the images) into account in the assessment. Primarily when follow-up exposures are made for monitoring the course of an illness, the follow-up exposures can in turn exhibit a different slice direction in comparison to prior exposures. Comparisons of the follow-up exposures with prior exposures thus can be made only with difficulty, since the precise magnitude of the different slice direction cannot be recognized in the image without further measures and therefore can be overlooked by a user. Differences that are actually due to the deviating slice direction can incorrectly be, for example, attributed to a progress of the illness.

The methods of United States Patent Application Publication No. 2003/139659 A1, DE 199 43 404 A1 and U.S. Pat. No. 6,195,409 allow the slice positioning to be determined before an acquisition to be implemented, such that a following acquisition is implemented with correct slice positioning. These methods, however, must be specially implemented in the acquisition system, which can be done only in rare cases. When a 3D volume data set 5 with a non-optimal positioning of the patient 9 has already been acquired, the methods offer no possibility of retroactive correction.

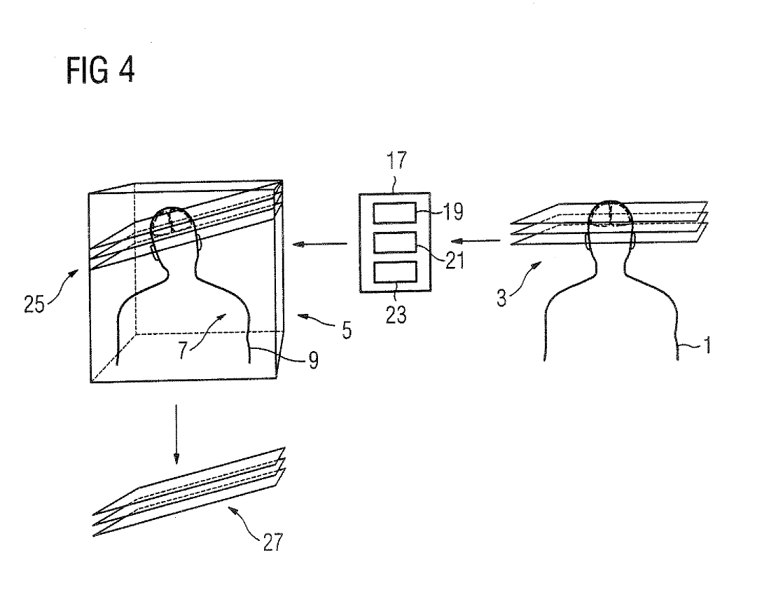

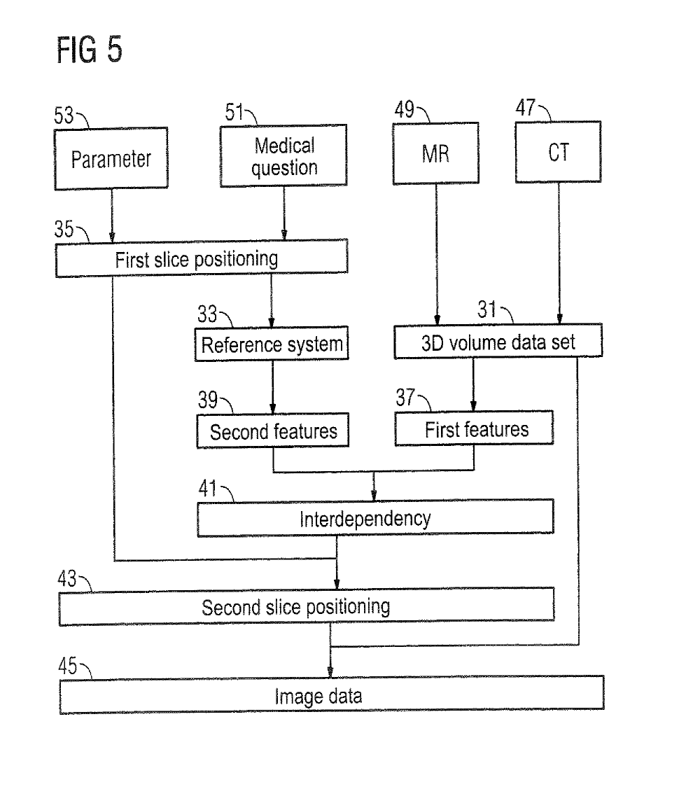

FIG. 3 and FIG. 4 show basic features of the inventive method; these features and their relation to one another being schematically shown again in FIG. 5.

First characteristic features 13 are initially extracted from the image 7. As indicated in FIG. 3, such characteristic features 13 can be anatomical landmarks that are easy to locate and that advantageously have a localization that does not vary too significantly between individuals.

Second characteristic features 15 that correspond to the first features 13 are also extracted in an analogous manner from the reference body 1.

The first and the second features 13, 15 are now set in relation to one another. From this a transformation 17 is derived that describes the relation between the image 7 and the reference body 1 and with which the reference body 1 and the image 7 can be transformed between one another.

As schematically indicated, such a transformation 17 can proceed based on different types of transformations.

For example, rigid transformations 19 describe a simple type of relation in which the reference body 1 and the image 7 are merely set in relation to one another via a rotation and/or a displacement. Affine transformations 21 furthermore take into account distortions and dilations. Going further, non-linear transformations 23 can more precisely detect differences between the reference body 1 and the image 7 in a spatially-dependent manner and significantly deform and distort the image 7 or the reference body 1 differently in a spatially-dependent manner.

The selected type of transformation 17 is thereby adapted to the medical question and the organ system to be imaged and represents a compromise between precision of the relation and calculation time for determination of the relation. For organ systems with a low inter-individual variability it can be adequate, for example, to merely determine a rigid or affine transformation 19, 21 that sets the image 7 and the reference body 1 in relation to one another in a best possible manner. In the case of other organ systems (for example, extremities) that can be bent differently in an image, non-linear transformations 23 are necessary in order to set the image 7 and the reference body 1 in relation to one another. If fixtures for the organs (for example for the head or an extremity) are used in the acquisition, the image of the organ will hereby exhibit a largely matching position so that only a simpler transformation is necessary in order to set it relative to a reference body.

The first and second features 13, 15 that are respectively extracted from the image and from the reference body and that form the basis for the transformation 17 to be determined, do not necessarily have to be anatomical landmarks as indicated in this exemplary embodiment. For example, intensity distributions in a 3D volume data set (for example the intensity distributions of the individual slice images) can also serve as features that are set in relation to intensity distributions in the reference body, in order to determine the transformations 17 therefrom that best converts the image 7 and the reference body 1 into one another. If the 3D volume data set 5 and the reference body 1 should additionally exhibit different contrasts (for example since the 3D volume data set and the reference body have been acquired with different MRT sequences), the transformation 17 is augmented such that these contrast differences are also taken into account.

Moment-based methods can likewise be used for specific images in order to determine a transformation 17 between reference body 1 and image 7. These methods use the intensity value distribution in the image in order to calculate corresponding abstracted quantities from this, similar to the calculation of diverse identifying values of a mass distribution such as a center of gravity or principle axes of inertia. Two varying images thus can be correlated in a simple manner by the transformation being calculated from the abstracted values.

After the matching transformation 17 has been determined, the advantageous first slice positioning 3 that is defined at the reference body 1 is adapted to the image 7 stored in the 3D volume data set 5 with the aid of the determined transformation 17, as is shown in FIG. 4.

In this manner a second slice positioning 25 is obtained that, in the 3D volume data set 5, now lies in a position corresponding to the first slice positioning 3. New two-dimensional views 27 of the organ to be images are now generated along the second slice positioning 25. The method used for this is advantageously a multiplanar reformatting (MPR).

Depending on the type of the transformation 17 (for example given non-linear transformations 23) it can also occur that the second slice positioning 25 comprises curved planes. The two-dimensional views 27 are then generated with an MPR adapted to the curved planes (what is known as “curved MPR”).

The two-dimensional views 27 now show the organ in the same advantageous orientation as was provided by the first slice positioning 3 at the reference body 1.

Particularly when follow-up acquisitions are executed or when exposures that were produced at different points in time are compared, it is possible with the method described herein to always obtain two-dimensional views 27 that show the organ in a view that corresponds to the first slice positioning 3 at the reference body 1, even if the patient 9 does not always have the same position in the acquisition of the 3D volume data set 5. A direct comparison of exposures that were produced at different points in time is thus enabled.

FIG. 5 again summarizes the essential features of the method and shows further features that are optional and give the method an additional flexibility or, advantageous development.

The starting point of the method is a 3D volume data set 31 in which an image of a subject is stored. A reference system 33 that represents the subject in an idealized form stands in relation to the 3D volume data set 31. A first slice positioning 35 is defined at this reference system 33.

Respective corresponding first features 37 and second features 39 are extracted from the 3D volume data set 31 and from the reference system 33, which first or, respectively, second features 37 or, respectively, 39 are set in relation to one another in order to obtain the interdependency 41 between the 3D volume data set 31 and the reference system 33.

This interdependency 41 is used in order to obtain from the first slice positioning 35 (which is defined at the reference system 33) a second slice positioning 43 that corresponds to the first slice positioning 35 in the 3D volume data set 31. Image data 45 that show the acquired subject in standardized views are acquired from the 3D volume data set 31 using the second slice positioning 43.

The 3D volume data set 31 is advantageously acquired with a computed tomography apparatus 47 or an MRT apparatus 49, but the method can also be applied when the image data set 31 has been acquired in a different manner, for example with a 3D ultrasound modality or a PET modality,

The method is advantageously implemented as a computer program in the computer of the apparatus with which the 3D volume data set 31 is also acquired.

In an embodiment the first slice positioning 35 that is defined at the reference system 33 can be selected from a number of possible slice positionings dependent on the medical question 51. For example, a user can input the medical question with which the first slice positioning 35 is then established. In a further advantageous embodiment the user can modify the first slice positioning 35 by input of parameters 53.

Particularly for systems that have been fitted with the feature of automatic slice positioning before an acquisition to be executed, the slice positionings stored there can also be used for the inventive method. In this manner, acquisitions that have already been executed that were acquired without an automatic slice positioning can be adapted to follow-up exposures that are acquired with the automatic slice positioning.

The disclosed method is not limited to medical imaging, but can also be applied to any imaging in which 3D volume data sets of subjects are produced.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for virtual slice positioning in a 3D volume data set representing an image of a subject, comprising the steps of:
from a 3D volume data set representing an image of a subject, extracting first image features associated with said subject;
automatically electronically determining an interdependency between said 3D volume data set of the subject and a reference system corresponding to the 3D volume data set, by setting the extracted first image features in relation to corresponding second image features in said reference system;
automatically electronically translating a first slice positioning, that is predefined at the reference system, to a second slice positioning in the 3D volume data set using said interdependency; and
generating image data from said 3D volume data set according to said second slice positioning.

2. A method as claimed in claim 1 comprising employing, as said 3D volume data set, a 3D volume data set representing a subject selected from the group consisting of a human, an animal, a body part of a human, and a body part of an animal.

3. A method as claimed in claim 1 comprising mathematically describing said interdependency as a transformation selected from the group consisting of rigid transformations, affine transformations, and non-linear transformations.

4. A method as claimed in claim 1 comprising determining said interdependency by an automatic electronic comparison of characteristic landmarks in said 3D volume data set and in said reference system.

5. A method as claimed in claim 1 comprising determining said interdependency by an automatic electronic comparison of intensity distributions respectively in said 3D volume data set and in said reference system.

6. A method as claimed in claim 1 comprising generating said image data according to said second slice positioning by multi-planar reformatting.

7. A method as claimed in claim 1 comprising establishing said predefined first slice positioning in said reference system dependent on a medical question.

8. A method as claimed in claim 1 comprising manually modifying said predefined first slice positioning by manual input of parameters at said reference system.

9. A method as claimed in claim 1 comprising acquiring said 3D volume data set with an imaging modality selected from the group consisting of computed tomography apparatuses and magnetic resonance apparatuses.

10. A computerized system for virtual slice positioning in a 3D volume data set representing an image of a subject, comprising:
a computer supplied with a 3D volume data set representing an image of a subject, that extracts first image features associated with said subject, and automatically determines an interdependency between said 3D volume data set of the subject and a reference system corresponding to the 3D volume data set, by setting the extracted first image features in relation to corresponding second image features in said reference system, and that automatically translates a first slice positioning, that is predefined at the reference system, to a second slice positioning in the 3D volume data set using said interdependency, and that generates image data from said 3D volume data set according to said second slice positioning; and
a display in communication with said computer at which said image data are visually presented.

11. A computerized system as claimed in claim 10 wherein said computer employs, as said 3D volume data set, a 3D volume data set representing a subject selected from the group consisting of a human, an animal, a body part of a human, and a body part of an animal.

12. A computerized system as claimed in claim 10 wherein said computer mathematically describes said interdependency as a transformation selected from the group consisting of rigid transformations, affine transformations, and non-linear transformations.

13. A computerized system as claimed in claim 10 wherein said computer determines said interdependency by an automatic electronic comparison of characteristic landmarks in said 3D volume data set and in said reference system.

14. A computerized system as claimed in claim 10 wherein said computer determines said interdependency by an automatic electronic comparison of intensity distributions respectively in said 3D volume data set and in said reference system.

15. A computerized system as claimed in claim 10 wherein said computer generates said image data according to said second slice positioning by multi-planar reformatting.

16. A computerized system as claimed in claim 10 wherein said computer establishes said predefined first slice positioning in said reference system dependent on a medical question.

17. A computerized system as claimed in claim 10 comprising an input unit allowing manual modification of said predefined first slice positioning by manual input of parameters at said reference system.

18. A computerized system as claimed in claim 10 comprising an imaging modality selected from the group consisting of computed tomography apparatuses and magnetic resonance apparatuses, that acquires said 3D volume data set.
